(19) United States
(12) Patent Application Publication
(10) Pub. No.: US 2011/0009684 A1
(43) Pub. Date: Jan. 13, 2011

(54) MULTI-STAGE MEMBRANE SEPARATION PROCESS

(75) Inventors: Zaida Diaz, Katy, TX (US); Henricus Abraham Geers, Rijswijk (NL); Ewout Martijn Van Jarwaarde, Amsterdam (NL); Arian Nijmeijer, Amsterdam (NL); Eric Johannes Puik, Rijswijk (NL)

Correspondence Address: SHELL OIL COMPANY, P O BOX 2463, HOUSTON, TX, 772522463, US

(73) Assignee: SHELL INTERNATIONALE RESEARCH MAATSCHAPPIJ B.V., Hague (NL)

(21) Appl. No.: 12/811,791

(22) PCT Filed: Jan. 7, 2009
(86) PCT No.: PCT/EP2009/050095

§ 371 Date: Sep. 29, 2010

Related U.S. Application Data
(60) Provisional application No. 61/019,664, filed on Jan. 8, 2008.

(51) Int. Cl. C07C 7/144

(52) U.S. Cl. 585/818

(57) ABSTRACT

The invention concerns a process for the removal of gaseous acidic contaminants, especially carbon dioxide and/or hydrogen sulphide, in two or more stages from a gaseous hydrocarbonaceous feedstream (1) comprising hydrocarbons and said acidic contaminants, using one or more membranes in each separation stages. The gaseous hydrocarbonaceous feedstream is especially a natural gas stream. The process is especially suitable for feedstreams comprising high amounts of acidic contaminants, e.g. between 10 and 95 vol. % of carbon dioxide and/or hydrogen sulphide, especially between 15 and 70 vol. %. In a first stage (2) a clean or almost clean hydrocarbon stream (3) is separated from the feedstream, the hydrocarbon stream suitably containing less than 5 vol % of acidic contaminants. The remaining stream (4) comprises the acidic contaminants and a certain amount of hydrocarbons. In a second stage (6) a pure or almost pure stream of acidic contaminants (8) is separated from the remaining stream (7), where after the then remaining stream is combined with the feed for the first stage (1), the acidic contaminants stream suitably containing less than 5 vol % of hydrocarbons.

### FIELD OF THE INVENTION

The present invention concerns a process for the removal of gaseous acidic contaminants, especially carbon dioxide and/or hydrogen sulphide, in two or more stages from a gaseous hydrocarbonaceous feedstream comprising hydrocarbons and said acidic contaminants, using one or more membranes in each separation stages.

### BACKGROUND

Natural gas is a major energy source. Its importance has increased in the past decades, and it is expected that its significance will grow further in the next decades. A main concern in the natural gas production is the presence of acidic contaminants. Many natural gas fields are known that contain a few percents of acidic contaminants, and many gas fields are known to comprise large amounts of acidic contaminants, e.g. between 10 and 50 vol % or sometimes even more, e.g. up till 90 vol %. In general, the presence of several volume percents of carbon dioxide and/or hydrogen sulphide will not create big problems, as conventional technologies are known to remove such amounts of acidic contaminants from the hydrocarbon fraction. Suitable conventional techniques are the absorption of acidic contaminants with aqueous amine solutions or with cold methanol, ethylene glycol dimethyl ether (DME) or polyethylene glycol, including the regeneration of the absorption liquids. The removal of higher amounts of acidic contaminants from natural gas, e.g. 10 vol percents or more, would result in very large removal units, including many stages, requiring very high investment and operational costs.

Thus, there is a need for new techniques for the easy and quick removal of acidic contaminants from natural gas streams containing high mounts of these compounds. In the past, the use of membranes has been considered for the removal of the acidic contaminants. However, up till now no process has be developed for the quick and easy removal of acidic contaminants from natural gas streams containing high mounts of these compounds.

### SUMMARY OF THE INVENTION

The present invention, now, describes an integrated multistage process for the removal of acidic contaminants from natural gas using two or more membranes stages, the membranes having a (much) higher permeance for the acidic components than for hydrocarbons, especially methane. In a first stage relative pure natural gas is obtained by removing all or almost all of the acidic components from the natural gas stream. The acidic contaminants containing stream, however, will contain a considerable amount of hydrocarbons, especially methane. In a second step, a pure or almost pure acidic contaminants containing stream is extracted from the acidic contaminants containing stream obtained in the first stage. The remaining stream from the second stage, containing hydrocarbons as well as acidic contaminants, is recycled to the natural gas feed stream that is used for the first stage.

In the above way, two streams are obtained, one stream a clean or almost clean natural gas stream, the other stream a clean or almost clean acidic contaminants containing stream. The first stream, optionally after further purification using conventional means, is suitably used as pipeline gas, or is used for the production of LNG or synthesis gas, for instance to be used as feedstream for the production of hydrogen, hydrocarbons (Fischer-Tropsch), methanol, urea etc. The second stream, may be used for instance for the production of sulphur or sulphur compounds, or may be used in an enhanced oil recovery (EOR) process.

Thus, the present invention concerns a process for the removal of gaseous acidic contaminants from a gaseous hydrocarbonaceous feedstream comprising such gaseous acidic contaminants, the process comprising: p1 1) providing the hydrocarbonaceous feedstream at a pressure between 30 and 120 bara,
2) contacting the feedstream with a membrane to obtain a hydrocarbon rich retentate and an acidic contaminants rich permeate,
3) optionally compressing the permeate obtained in step 2) up till a pressure between 30 and 120 bara,
4) contacting the compressed permeate with a second membrane to obtain a hydrocarbon rich retentate and an acidic contaminants rich permeate,
5) optionally compressing the hydrocarbon rich retentate up till a pressure between 30 and 120 bara, and
6) mixing the hydrocarbon retentate obtained in step 5) with the feedstream of step 1), with the proviso that steps 3 and 5 comprise at least one compressing stage.
   The gaseous hydrocarbonaceous feedstream is especially a natural gas stream. The process is especially suitable for feedstreams comprising high amounts of acidic contaminants, e.g. between 10 and 95 vol. % of carbon dioxide and/or hydrogen sulphide, especially between 15 and 70 vol. %. In a first stage a clean or almost clean hydrocarbon stream is separated from the feedstream, the hydrocarbon stream suitably containing less than 5 vol. % of acidic contaminants. The remaining stream comprises the acidic contaminants and a certain amount of hydrocarbons. In a second stage a pure or almost pure stream of acidic contaminants is separated from the remaining stream, where after the then remaining stream is combined with the feed for the first stage, the acidic contaminants stream suitably containing less than 5 vol % of hydrocarbons.

### DETAILED DESCRIPTION

The process of the invention separates acidic contaminants containing hydrocarbons streams, especially natural gas stream, into two relatively pure streams, one hydrocarbon stream and an acidic contaminants containing stream. The process uses relatively cheap membranes. Membrane units, when compared with conventional treating processes as amine absorption including regeneration, require a relatively small operational area, require small amounts of energy, and require only little operational efforts. Also maintenance and inspection requirements are moderate.

The feedstream for the process of the invention will have a pressure between 30 and 120 bara. Especially, the feedstream has a pressure between 40 and 100 bara, preferably between 50 and 90 bara. The feedstream suitably has a temperature between −30 and 120° C., suitably between −20 and 100° C., preferably between 0 and 50° C.

The acidic contaminants in the feedstream are especially carbon dioxide and hydrogen sulphide, although also carbonyl sulphide (COS), carbon disulphide (CS2), mercaptans, sulphides and aromatic sulphur compounds may be present. Beside acidic contaminants, also inerts may be present, for instance nitrogen and noble gases as argon and helium, usually in an amount up till 20 vol %, especially up till 10 vol %.

The amount of acidic contaminants in the gaseous hydrocarbonaceous feedstream may vary within a broad range. Suitably, the amount of carbon dioxide is between 10 and 95 vol % based on the total feedstream, preferably between 15 and 75 vol %, e.g. for gaseous hydrocarbonaceous feedstream from subsurface reservoirs, or between 80 and 95 vol %, e.g. from specific recycle streams, especially EOR recycle streams. The amount of hydrogen sulphide is suitably between 0 and 45 vol % based on the total feedstream, preferably between 5 and 40 vol %.

The amount of hydrocarbons in the gaseous hydrocarbonaceous feedstream may vary within a broad range. Suitably, the feedstream comprises hydrocarbons in an amount between 5 and 90 vol % based on total feedstream, preferably between 5 and 15 vol %, e.g. for recycle streams as EOR recycle stream, or between 20 and 90 vol %, for instance for feedstreams produced from subsurface natural gas reservoirs. The hydrocarbons in the feedstream usually will contain large amounts of methane, suitably between 50 and 98 vol %, especially 60 and 95 vol %, based on the volume of the total feedstream.

Membranes to be used in the process of the present invention are known in the literature. It is advantageous to use membranes with a high selectivity for acidic contaminants as carbon dioxide and hydrogen sulphide. The selectivity is defined as the ratio of the acidic contaminants permeability over the permeability of the hydrocarbons as measured in single gas experiments. Preferably, the selectivity of the membrane in step 2) is between 10 and 200, preferably between 20 and 150.

The permeance for carbon dioxide or hydrogen sulphide of the membrane in step 2) is suitably between 10⁻¹⁰ and 10⁻⁴ mol/m2sPa, preferably the carbon dioxide or hydrogen sulphide permeance through the membrane in step 2) is between 10⁻⁹ and 10⁻⁵ mol/m2sPa.

The permeate obtained in step 2) suitably has a pressure between 1 and 30 bara, preferably between 5 and 25 bara. The retentate obtained in step 2) will have a pressure more or less the same as the pressure of the gaseous hydrocarbonaceous feedstream. Suitably the retentate obtained in step 2) has a pressure which is up till 5% less than the pressure of the feedstream, preferably up till 2% less.

The retentate obtained in step 2 suitably has a hydrocarbon content of >95 vol % based on the total retentate stream, preferably more than 97 vol %. It is observed that the person skilled in the art by variation of e.g. the permeance of the membrane, the contact area of the membrane and the contact time with the membrane is able to vary the purity of the retentate obtained in step 2). Suitably, the retentate in step 2) has an acidic contaminants content of less than 2 vol % based on the total retentate, preferably less than 1 vol %.

The permeate stream obtained in step 2) of the process of the present invention will contain beside the acidic contaminants, also a relatively large amount of hydrocarbons. This is due to the fact that removal of all or almost all acidic contaminants, also will result in a relatively large amount of hydrocarbons to pass through the membrane. In general it can be said that the more pure the hydrocarbon containing stream will be, the more hydrocarbons will be present in the permeate. Suitably, the permeate in step 2) has a carbon dioxide or hydrogen sulphide content of between 25 and 90 vol % based on the total permeate stream, preferably between 40 and 80 vol %.

The membrane to be used in step 2) of the process of the present invention may be any membrane known in the art, provided that it will have a clear selectivity for acidic contaminants. Suitably the membrane is chosen from a polyethylene oxide based membrane, preferably a polyethylene oxide based membrane comprising block-copolymers, especially PEO 600/5000 T6T6T or a cross linked PEO, a polyimide or polyaramide based membrane, a cellulose acetate based membrane, a zeolite based membrane, preferably a silica-alumina phosphate based membrane, especially, SAPO-34, a micro-porous silica membrane or a carbon molecular sieves membrane.

The membrane in step 4) may be the same membrane as used in step 2). Suitably the selectivity of the membrane in step 4) is between 10 and 200, preferably between 20 and 150.

The permeance for carbon dioxide or hydrogen sulphide of the membrane in step 4) is suitably between 10⁻¹⁰ and 10⁻⁴ mol/m2sPa, preferably the carbon dioxide or hydrogen sulphide permeance through the membrane in step 2) is between 10⁻⁹ and 10⁻⁵ mol/m2sPa.

The permeate obtained in step 4) suitably has a pressure between 1 and 20 bara, preferably between 5 and 10 bara. The retentate obtained in step 4) will have a pressure more or less the same as the pressure of the feedstream. Suitably the retentate obtained in step 4) has a pressure that is up till 5% less than the pressure of the feedstream, preferably up till 2% less.

The permeate obtained in step 4) suitably has a carbon dioxide or hydrogen sulphide content of more than 80 vol % based on total retentate stream, preferably more than 90 vol %, more preferably more than 98 vol %. Preferably the permeate in step 4) contains less than 3 vol % of hydrocarbons, preferably less than 1 vol %. It is observed that the person skilled in the art by e.g. variation of e.g. the permeance of the membrane, the contact area of the membrane and the contact time with the membrane is able to vary the purity of the permeate obtained in step 2). Suitably the retentate in step 4) has a hydrocarbon content of between 40 and 90 vol % based on the total retentate stream, preferably between 50 and 80 vol %.

The membrane to be used in step 4) of the process of the present invention may be any membrane known in the art, provided that it will have a clear selectivity for acidic contaminants. Suitably the membrane is chosen from the same membrane categories as defined above for step 2).

In the process of the invention, the permeate of step 3) and/or the permeate of step 5) needs to be compressed to a pressure between 30 and 120 bara. In that way the permeate obtained in step 5) can be mixed with the feed for step 1). Preferably the permeate obtained in step 5, after compression after step 2 and/or step 4), has a pressure equal to the pressure of the feed for step 1). Preferably only the permeate of step 2 is compressed to the required pressure.

In a preferred embodiment the process of the present invention comprises obtaining the gaseous hydrocarbonaceous feedstream from a gaseous feed comprising hydrocarbons and acidic contaminants by contacting the gaseous feed with a membrane to obtain the feedstream and an acidic contaminants rich permeate. In this way the process of the present invention is preceded by a bulk separation of acidic contaminants. The acidic contaminants are especially one or more compounds selected from carbon dioxide and hydrogen sulphide. By choosing the conditions in an optimum way, a permeate will be obtained containing high or very high amounts of acidic contaminants. Suitably, the permeate has a carbon dioxide and hydrogen sulphide content of more than 90 vol %, preferably more than 96 vol %. The membrane to be used in this additional step may be any membrane known in the prior art, provided that it will have a clear selectivity for acidic contaminants, e.g. a selectivity of 5 or higher. Suitably the membrane is chosen from the same membrane categories as defined above for step 2). In the additional step the permeate suitably has a pressure between 1 and 30 bara, preferably between 5 and 15 bara. The selectivity of the membrane in the additional step is suitably between 10 and 200, preferably between 20 and 150.

The permeance for carbon dioxide or hydrogen sulphide of the membrane in the additional step is suitably between 10⁻¹⁰ and 10⁻⁴mol/m2sPa, preferably the carbon dioxide or hydrogen sulphide permeance through the membrane in step 2) is between 10⁻⁹ and 10⁻⁵ mol/m2sPa.

The feed for the additional step suitably has a pressure between 30 and 120 bara. Especially, the feed has a pressure between 40 and 100 bara, preferably between 50 and 90 bara. The feed suitably has a temperature between −30 and 100° C., suitably between −20 and 70° C., preferably between 0 and 50° C. The retentate in this step will have a pressure more or less the same as the pressure of the gaseous feed. Suitably the feed has a pressure up till 5% less than the pressure of the feedstream, preferably up till 2% less. The permeate suitably contains less than 10 vol % of hydrocarbons, preferably contains less than 3 vol % hydrocarbons, more preferably less than 1 vol %.

The carbon dioxide and/or hydrogen sulphide rich permeate obtained in step 4) of the process of the invention and/or in the additional step may be used for instance for enhanced oil recovery. In that case the permeate of step 4) or of the additional step is suitably recompressed up till a pressure suitably between 80 and 400 bara, especially between 150 and 300 bara. Preferably the retentate obtained in the additional step is combined with the retentate obtained in step 4), preferably followed by compression.

The invention further relates to the use of the compressed carbon dioxide and hydrogen sulphide rich permeates produced in one or more processes of the invention in enhanced oil recovery.

The invention also relates to the use of the hydrocarbon rich retentate produced in one or more processes of the invention as pipeline gas, LNG feed or GTL feed.

A preferred embodiment of the process of the present invention comprises a pretreatment of the gaseous carbonaceous feedstream or the gaseous feed in order to remove water. This is suitably done by a glycol treatment, for instance using MEG, DEG and/or TEG, a glycerol treatment or a molsieve treatment. Further, the process may also comprise the removal of hydrocarbons higher than methane, preferably at least the C5+ fraction, more preferably also the C2-C4 fraction, before the carbon dioxide and/or the hydrogen sulphide is removed.

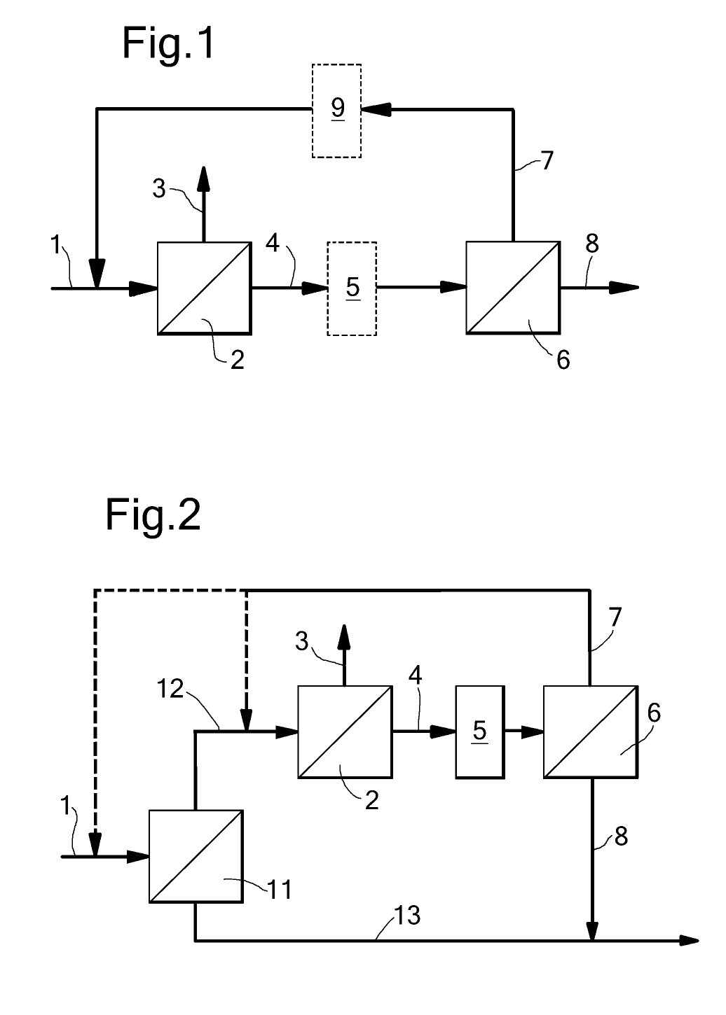

The invention is described in a non-limiting manner in FIGS. 1 and 2.

In FIG. 1 a dried, gaseous hydrocarbonaceous feedstock (pressure 100 bar, temperature 20° C., 55 vol % CO2) is contacted with a membrane in unit 2. An almost pure stream of hydrocarbons (pressure 98 bar, 2 vol % CO2) is removed from unit 2 via line 3. A permeate (pressure 20 bar, 85 vol % CO2) is removed via line 4. The permeate may be compressed in unit 5. The permeate is contacted with a second membrane in unit 6. An almost pure stream of carbon dioxide (98 vol %) is removed via line 8. The retentate stream, a mixture of hydrocarbons and carbon dioxide, is removed via line 7. The retentate may be compressed in unit 9. It is observed that there is either a compression step in unit 5 or in unit 9. The retentate from unit 6 is mixed with original feedstream 1.

In FIG. 2 a dried gaseous hydrocarbonaceous feedstream comprising carbon dioxide and hydrogen sulphide is contacted with a membrane in unit 11 to separate carbon dioxide and hydrogen sulphide from a hydrocarbon enriched retentate stream 12. This stream is treated in the same way as described in FIG. 1. The retentate stream 7 from unit 6 may be recirculated to either unit 2, or, preferably, to unit 11. The permeate streams 13 from unit 11 and 8 from unit 6 are combined. In this scheme an optimum removal of acidic components is obtained. Only one compressing unit is necessary.

1. A process for the removal of gaseous acidic contaminants from a gaseous hydrocarbonaceous feedstream comprising one or more gaseous acidic contaminants, the process comprising:
providing the hydrocarbonaceous feedstream at a pressure between 30 and 120 bara;
contacting the feedstream with a membrane to obtain a hydrocarbon rich retentate and an acidic contaminants rich permeate;
compressing the acidic contaminants rich permeate up till a pressure between 30 and 120 bara;
contacting the permeate with a second membrane to obtain a second hydrocarbon rich retentate and a second acidic contaminants rich permeate;
compressing the hydrocarbon rich retentate up till a pressure between 30 and 120 bara; and
mixing the second hydrocarbon rich retentate with the hydrocarbonaceous feedstream rich.

2. The process according to claim 1, in which the feedstream has a temperature between −20 and 100° C.

3. The process according to claim 1, in which the acidic contaminants comprise one or more compounds selected from carbon dioxide and hydrogen sulphide.

4. The process according to claim 3, in which the feedstream comprises carbon dioxide in an amount between 10 and 95 vol % based on the total feedstream volume.

5. The process according to claim 3, in which the feedstream comprises hydrocarbons in an amount between 5 and 90 vol % based on total feedstream volume.

6. The process according to claim 1, in which the acidic contaminant rich permeate has a pressure between 1 and 30 bara.

7. The process according to claim 1, in which the hydrocarbon rich retentate has a hydrocarbon content of >95 vol % based on the total retentate stream volume.

8. The process according to claim 1, in which the acidic contaminants rich permeate has a carbon dioxide content of between 40 and 80 vol % based on the total permeate stream.

9. The process according to claim 1, in which the second acidic contaminants rich permeate has a pressure between 1 and 20 bara.

10. The process according to claim 1, in which the second hydrocarbon rich retentate has a hydrocarbon content of between 40 and 90 vol % based on the total retentate stream.

11. The process according to claim 1, in which the second acidic contaminants rich permeate has a hydrogen sulphide content of more than 90 vol %.

12. The process according to claim 1, in which the second acidic contaminants rich permeate contains less than 3 vol % of hydrocarbons.

13. A process according to claim 1, in which the process further comprises obtaining the gaseous hydrocarbonaceous feedstream from a gaseous feed comprising hydrocarbons and acidic contaminants by contacting the gaseous feed with a membrane to obtain the feedstream and an acidic contaminants rich permeate.

14. (canceled)

15. A process according to claim 1, further comprising a pretreatment of the hydrocarbonaceous feedstream in order to remove water by a glycol treatment, a glycerol treatment, a molsieve, or silica gel treatment.

16. The process according to claim 1, wherein compressing the acidic contaminants rich permeate comprises increasing a pressure to a valve between 30 and 120 bara.

17. The process according to claim 1, wherein compressing the hydrocarbon rich retentate comprises increasing a pressure to a valve between 30 and 120 bara.
